(19) United States
(12) Patent Application Publication
(10) Pub. No.: US 2006/0111787 A1
(43) Pub. Date: May 25, 2006

(54) Glenoid prosthesis and method of implanting same

(76) Inventors: David S. Bailie, Scottsdale, AZ (US); Marc A. Riner, Aristau (CH)

Correspondence Address: KNOBBE MARTENS OLSON & BEAR LLP, 2040 MAIN STREET, FOURTEENTH FLOOR, IRVINE, CA, 92614, US

(21) Appl. No.: 11/269,125

(22) Filed: Nov. 7, 2005

(30) Foreign Application Priority Data
Nov. 5, 2004 (DE) 10 2004 053 606.6

(51) Int. Cl. A61F 2/40

(52) U.S. Cl. 623/19.13; 623/908

(57) ABSTRACT

A glenoid prosthesis includes a bearing, the lateral side of which defines a concave bearing surface, and a carrier shell, the medial side of which comprises anchoring elements, wherein the medial side is generally flat. The prosthesis may be implanted in an anterior-posterior (A-P) by forming bores in the scapula in the A-P direction, wherein the anchoring elements extend into the bores. The scapula may be proceed to have a generally flat surface to correspond with the flat surface at the medial side of the prosthesis.

### CROSS-REFERENCE TO RELATED APPLICATIONS

This application claims priority to German Application No. DE 10 2004 053 606.6, filed Nov. 5, 2004, the entire contents of which are incorporated herein by reference and should be considered a part of this specification.

### BACKGROUND OF THE INVENTION

1. Field of the Invention

The invention relates to a glenoid prosthesis, and in particular relates to a prosthetic joint replacement for a shoulder joint, with a bearing shell the medial side of which, where appropriate, comprises anchoring elements and the lateral side of which defines a concave bearing surface.

2. Description of the Related Art

Conventional shoulder joint replacement includes the replacement of either only the joint surface on the humerus side, i.e. the head of the humerus (hemi-arthroplasty), or the replacement of both joint surfaces, i.e. the head of the humerus and the glenoid fossa (total arthroplasty). Both monoblock and modular endoprostheses can be employed in shoulder joint replacement procedures.

The artificial joint replacement is usually made of metal. In particular, the artificial joint can be made of a cobalt-chromium or titanium alloy, or of pure titanium. However, ceramics and polyethylene (PE) can also be used. The implants on the humerus side are either cemented into place or installed without cement. The same applies to the scapular-side implant, which ac s as the slide partner to the artificial humerus head. The artificial joint also includes a bearing structure, usually made of polyethylene, with a bearing surface opposite the humerus head.

Modular artificial joint systems generally comprise a carrier shell for a bearing structure incorporating the bearing surface. The carrier shell is generally made of metal, in particular titanium or titanium alloy, and can include anchoring means in the form of “pegs” or “keels”, with which to anchor the shell in the bone. Alternatively, the carrier shell can also be anchored in the bone by means of screws, adjusting screws or expandable pegs. Even when cement is used, anchoring means can be provided on the back surface of the implant to ensure better retention in the cement. Glenoid prostheses known in the art are further described, for example, in EP 0 903 127 A2, EP 1 013 246 A1 and EP 1 136 046 A2, the entirety of each of which is hereby incorporated by reference.

However, conventional artificial joint prostheses have a back surface that is medially convex in order to permit the implant to be inserted only in a direction that is substantially perpendicular to the shoulder blade, i.e. scapula. This applies in particular when anchoring means, in particular anchoring pegs, are also disposed on the medial side, wherein the anchoring means extend perpendicular thereto. As a result, additional processing (e.g., drilling, milling) is required in a direction parallel to the normal line of the original glenoid, for which a generous amount of space is a prerequisite. Such work can be done only by experienced operators.

Furthermore, with conventional implants there is a risk of the loosening of the glenoid prosthesis. If there is a mismatch between the radius of the ball (humerus-head replacement) and that of the socket (glenoid) due to the actual anatomy of the shoulder, such that the radius of the ball is smaller than the radius of the socket, a translational movement in the caudal-cranial direction becomes possible. This movement is limited by the edge of the socket (glenoid). When the ball pushes against the edge of the socket, a force is exerted on the socket that causes leverage out of the socket. This phenomenon is commonly known in the literature as the “rocking-horse” effect, and appears in both “keeled” and “pegged” implants.

### SUMMARY OF THE INVENTION

It is thus one objective of the present invention to provide a glenoid prosthesis that is easier to handle than conventional prostheses, and with which a processing parallel to the normal line of the joint surface (i.e., perpendicular to the joint surface) is replaced by a processing and also implantation in the anterior/posterior (A-P) direction. These advantages are intended to apply to both a cement-free and a cemented version of the prosthesis.

In accordance with one embodiment, a glenoid prosthesis is provided comprising a bearing, a lateral side of which defines a concave bearing surface. The prosthesis also comprises a carrier shell, a medial side of which comprises at least one anchoring element, wherein the medial side is generally flat.

In another embodiment, a glenoid prosthesis is provided comprising a lateral bearing surface and at least one anchoring element on a medial side of the prosthesis. The at least one anchoring element comprises a ridge extending generally in an anterior-posterior direction and an enlarged portion at a free end thereof. Optionally, the prosthesis may comprise a carrier shell comprising the at least one anchoring element and a bearing structure comprising the lateral bearing surface, wherein the carrier shell and the bearing structure are fixed to each other.

In accordance with another embodiment, a method for implanting a glenoid prosthesis is provided. The method includes positioning a cutting guide on an anterior surface of the glenoid of a scapula, and removing bone from the scapula at the glenoid to a subchondral position to form a generally flat surface. The method also includes positioning a drill guide on the scapula, drilling at least one bore in the scapula in generally an anterior-posterior direction, and forming at least one slot in communication with the at least one bore, the slot extending to the generally flat surface. The method further comprises attaching the glenoid prosthesis to the scapula, wherein the glenoid prosthesis includes a generally flat medial surface and at least one anchoring element projecting from the medial side and extending in generally an anterior-posterior direction, wherein the glenoid prosthesis is attached to the scapula by inserting the at least one anchoring element into the bore and the slot in an anterior-to-posterior direction. In one embodiment, the glenoid prosthesis has two anchoring elements. In another embodiment, a screw can be implanted in the anterior-to-posterior direction to facilitate fixation of the prosthesis. For example, the carrier shell can have a plate that, upon implantation, lies proximal the anterior glenoid neck, the plate defining a hole that allows a screw to be inserted therethrough. The hole can be threaded or unthreaded. In one embodiment, the screw can extend between two anchoring elements. In another embodiment, the screw can be inserted in association with one anchoring element.

In accordance with another embodiment, a method for implanting a glenoid prosthesis comprises forming a generally flat surface on a lateral side of a patient's scapula. A glenoid prosthesis is attached to the patient's scapula, the glenoid prosthesis having a generally flat surface on a medial side thereof to engage the generally flat surface on the lateral side of the patient's scapula. The glenoid prosthesis also has at least one anchoring element attaching the prosthesis to the scapula, and a bearing surface on a lateral side thereof.

In accordance with another embodiment, a method for implanting a glenoid prosthesis comprises forming at least one bore in generally an anterior-posterior direction in a patient's scapula, the bore communicating with a lateral side of the patient's scapula. A glenoid prosthesis is attached to the patient's scapula, the prosthesis having at least one anchoring element on a medial side thereof, wherein the prosthesis is attached by inserting the anchoring element through the at least one bore such that the medial side of the glenoid prosthesis engages the lateral side of the patient's scapula.

### BRIEF DESCRIPTION OF THE DRAWINGS

Embodiments of a glenoid prosthesis are described in greater detail below with reference to the attached drawings, wherein

FIG. 1 is a perspective side view of one embodiment of a glenoid prosthesis.

FIG. 2 is a perspective rear view of one embodiment of a carrier shell of the prosthesis according to FIG. 1.

FIG. 3 is a perspective side view of the carrier shell according to FIG. 1 and/or FIG. 2.

FIG. 4 is a perspective rear view of one embodiment of a bearing structure of the prosthesis according to FIG. 1.

FIG. 5 is a schematic front view of a conventional total prosthesis for a shoulder joint.

FIG. 6 is a schematic front view of a total prosthesis, including a glenoid prosthesis according to one embodiment of the present invention.

### DETAILED DESCRIPTION OF THE PREFERRED EMBODIMENTS

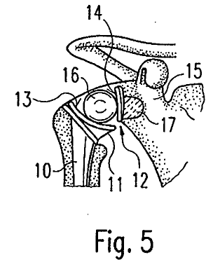

For better understanding of the context of the glenoid prosthesis in accordance with the invention, a shoulder-joint total prosthesis according to the state of the art will be described with reference to FIG. 5, which shows a humerus nail 10 with a bearing structure 11. The bearing structure 11 defines a concave bearing surface 13 for a joint or humerus ball 16. On the scapula side, the joint ball 16 is supported against a glenoid prosthesis 12, which has a bearing surface 14 that likewise has a concave curvature. The glenoid prosthesis 12 is anchored in the scapula, i.e. in the shoulder blade 15, where said anchoring is reinforced by an anchoring peg 17.

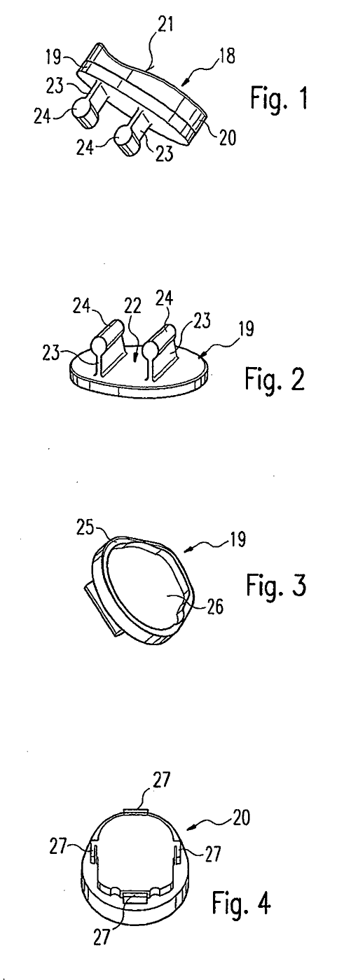

Embodiments of the present invention are concerned with further development of the glenoid prosthesis 12, as discussed with reference to FIGS. 1-4.

FIG. 1 shows a glenoid prosthesis 18 comprising a carrier shell 19 preferably made of a human-tissue-compatible metal, such as titanium or a titanium alloy, and a bearing structure 20 preferably made of plastic, such as polyethylene, a ceramic material or metal. However, the carrier shell 19 can also be made of a plastic material, such as a plastic-fiber composite, that exhibits a similar strength as metal. The bearing structure 20 is positioned on the lateral side of the implant and defines a concave bearing surface 21. The concave bearing surface 21 can comprise at least two spherical surfaces differing from one another in radius. Alternatively the bearing surface can be part of an ellipsoid, hyperboloid or a paraboloid surface. Preferably, the medial (i.e., back) side 22 of the implant (see FIG. 2), specifically of the carrier shell 19, is generally flat, and more preferably may be substantially or completely flat forming a planar area from which two ridges 23 spaced apart from one another project. Each of the ridges 23 extend in the anterior/posterior direction and has an expanded portion 24 at its free end. In the illustrated embodiment, the expanded portion 24 has a cylindrical shape with a round cross-section along a plane generally perpendicular to the anterior-posterior direction. In other embodiments, the cross-section of the expanded portion 24 can be rectangular, trapezoidal, or oval. The illustrated embodiment shows two ridges 23 extending from the medial side 22 of the carrier shell 19, each ridge 23 having an expanded portion 24 at a free end thereof. However, in another embodiment the carrier shell 19 can have one ridge 23 with an expanded portion 24 extending from the medial side 22 thereof. In still another embodiment, a screw can be implanted in an A-P direction to facilitate fixation of the prosthesis 18. For example, the carrier shell 19 can have a plate that, upon implantation, lies proximal the anterior glenoid neck, the plate defining a hole that allows the screw to be inserted therethrough. The screw can be threaded or unthreaded. In one embodiment, the screw can extend between two ridges 23. In another embodiment, the screw can be inserted in association with one ridge 23.

The embodiment illustrated in FIGS. 1-4 is modular. Specifically, the prosthesis 18 comprises a separate carrier shell 19, as well as a bearing structure 20 that attaches thereto and has a bearing surface 21. The bearing structure 20 is preferably fastened to the carrier shell 19 in a force-fitting and/or form-fitting manner. Additionally, the bearing structure 20 and carrier shell 19 can be fastened together in-situ or prior to implantation.

In the illustrated embodiment, the carrier shell 19 and bearing structure 20 are fastened together by a catch means. In another embodiment, the carrier shell 19 and bearing structure 20 can form part of a monoblock or one-piece glenoid prosthesis. In the illustrated embodiment, the lateral side of the carrier shell 19 is trough-shaped, as shown in FIG. 3 and comprises a circumferential rim 25 bordering a generally flat floor 26. The inside of the circumferential rim 25 preferably has at least one undercut portion sized to receive and engage a complementary projection 27 on the associated side of the bearing structure 20. Accordingly, in the illustrated embodiment, the catch means includes the undercut portions of the carrier shell 19 and the projections 27 of the bearing structure 20.

The undercut portions can include recesses, slots, grooves, or other configurations suitable for lockably engaging the projections 27. In one embodiment, the undercut portion can extend along the circumference of the rim 25. In another embodiment, the undercut portions can be formed at discrete locations about the circumference of the rim 25.

In the illustrated embodiment, a total of four projections 27 are provided on the underside of the bearing structure 20, which correspond to complementary undercuts on the inside of the circumferential rim 25 of the carrier shell 19. However, any number of suitable projections 27 can be provided to fasten the carrier shell 19 and bearing structure 20 together.

As shown in FIG. 4, the projections 27 are preferably equidistantly positioned around the periphery of the bearing structure 20. In another embodiment, the projections can be positioned at non-equidistant locations about the periphery of the bearing structure 20. However, any suitable mechanism for fastening the carrier shell and bearing structure can be used. As shown in FIG. 1, when assembled the circumferential surface of the bearing structure 20 is substantially flush with the outer circumference of the circumferential rim 25.

In the illustrated embodiment, the glenoid prosthesis 18 is modular in construction. In another embodiment, the glenoid prosthesis can also be formed as one piece, which can be made of plastic, ceramic or metal. Additionally, in one embodiment the carrier shell 19 and the bearing structure 20 are removably fastened to each other. In another embodiment, the bearing structure 20 is permanently attached to the carrier shell 19, so as to form a quasi-integral prosthesis.

Preferably, the glenoid prosthesis 18 shown in FIGS. 1-4 has an elongated oval shape, which can be formed using any suitable manner known in the art. However, the glenoid prosthesis 18 can have other shapes, such as circular.

In one embodiment, the prosthesis 18 comprises the use of cement. In such an embodiment, the medial side of the implant 18 corresponds to that of the medial side of the shell 19, and the lateral side of the implant corresponds to that of the lateral side of the bearing structure 20.

In one embodiment, where the bearing structure 20 is made of a ceramic or metallic material, the prosthesis 18 preferably includes an intermediate layer. The intermediate layer is preferably disposed between the carrier shell 19 and the bearing structure 20 and is made of a plastic, such as PE or PEEK.

The bores in the bone that correspond to the ridge expansions 24 are preferably formed with a drill guide, which can be positioned on an anterior surface of the scapula 15 with the bores formed by drilling in an anterior-posterior direction. In one embodiment, the drill guide can be fastened to the scapula with a k-wire. However, other suitable mechanisms can be used to fasten the drilling guide.

In one embodiment, a surgical procedure for implanting the glenoid prosthesis 18 comprises resecting bone from the scapula. Preferably, bone is resected to a subchondral position so as to provide a generally planar surface to correspond to the planar surface of the medial side 22 of the carrier shell 19. The drill guide described above may be used to produce the planar surface, or alternatively, a separate cutting guide may be used to resect bone, which may be attached to an anterior surface of the glenoid of the scapula with a K-wire. After resection of bone, a keyhole or drill guide may be positioned on an anterior surface of the scapula proximal the location of the glenoid, preferably fixed to the scapula with a K-wire. In one preferred embodiment, the drill guide has a generally L-shaped body and preferably includes at least one elongated bore formed on an anterior member of the body, more preferably two elongated bores, corresponding in size to the enlarged expansions 24 of the carrier shell 19. Adjacent each of the bores, on a lateral member of the body that extends generally perpendicular to the anterior member, slots may be provided in the drill guide corresponding in size to the ridges 23 of the carrier shell 19. The drill guide is preferably positioned so that the anterior member is adjacent an anterior surface of the scapula 15 and so the lateral member is adjacent the planar surface on the glenoid. Bores are then preferably drilled into the scapula in an anterior-to-posterior direction through the bores in the drill guide. Additionally, slots are preferably formed on the scapula through the slots in the drill guide, so that each bore drilled in the scapula communicates with a corresponding slot. In one embodiment, the slots are formed in an anterior-to-posterior direction. In another embodiment, the slots are formed in a lateral-to-medial direction on the planar surface of the glenoid through slots in the lateral member. In yet another embodiment, the slots are formed in both an anterior-to-posterior direction and a medial-to-lateral direction. These slots may be formed to extend from the bores to the planar surface on the lateral side of the scapula. The bores thus obtained may be connected by way of saw-cuts to the flattened bearing surface for the glenoid prosthesis 18.

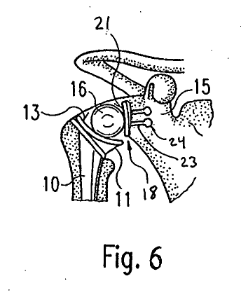

In one embodiment, said drill guide can also be simultaneously used as a cutting guide for preparation of the planar contact surface on the scapula for contact with the glenoid implant 18. The prosthesis 18 can then be implanted in an anterior-posterior direction, so that the ridge expansions 24 extend into the bores drilled in the scapula, and so the anchoring ridges extend through the slots connected to the bores, as shown in FIG. 6 (showing a single ridge embodiment).

The glenoid prosthesis 18 shown in FIGS. 1-4 is preferably anchored in bone in a form-fitting manner, so as to inhibit the leveraging out of the implant. Advantageously, the cylindrically-shaped ridge expansions 24 inhibit the “rocking-horse” effect discussed above. This applies to both the cemented and the cement-free versions of the implant 18.

In one preferred embodiment, the overall thickness of the prosthesis 18, i.e. of carrier shell 19 plus bearing structure 20, is between about 3.0 mm and 10.0 mm. In another embodiment, the overall thickness of the prosthesis 18 is about 5.0 mm to 7.5 mm. However, the prosthesis 18 can have other suitable thicknesses.

In one embodiment, the bearing structure 20 can be fastened to the carrier shell 19 using screws. In another embodiment, a dovetailed fastener arrangement can be used, so that the bearing structure 20 can be inserted into the carrier shell 19, from one side, whereupon the dovetailed fastener can engage the bearing structure 20 and carrier shell 19.

In one embodiment, the medial side of the carrier shell 19 is preferably roughened in any suitable manner known in the art in order to facilitate bone growth onto it. In one preferred embodiment, a grit-blasted, titanium plasma sprayed, Hydroxylapatite (HA) coating or other on/in-growth coatings used in joint replacement is applied to the carrier shell 19 to facilitate bone growth onto it. The medial side and/or the anchoring elements 24 also preferably comprise recesses (not shown) to enhance the process of bone growth into the prosthesis 18.

Advantageously, because the medial side of the glenoid prosthesis 18 is generally flat, more preferably substantially or completely flat and extending in the form of a plane, the processing of the scapular surface with a complementary shape is made correspondingly simple. Furthermore, the configuration of the medial side allows implantation in the anterior-posterior (A-P) direction, especially-when the anchoring elements on the medial side extend in the A-P direction. In the case of cement-free implantation, of course, the scapular side should also be correspondingly prepared. This is done by means of a bone-processing tool that preferably operates in the anterior/posterior direction. As a result, processing of the scapular side parallel to the normal line of the original glenoid can be avoided, and considerably less processing space is needed than with conventional prostheses, thus reducing the corresponding stress imposed on the patient.

In another embodiment, the prosthesis 18, and in particular the carrier shell 19, can also comprise means for fixation of ball heads. For example, a ball head or hemisphere may be attached to or integrally formed with the lateral side of the of the carrier shell to form a reverse shoulder prosthesis (e.g., a glenosphere). In particular, the bearing structure 20 as described above may have a spherical or hemispherical shape to form the reverse shoulder prosthesis. This ball head replaces the ball 16 shown in FIG. 6, and acts as a bearing surface against a humerus side bearing surface. Screws, pegs, adhesive, force-fitting or other suitable means may be used to attach the ball head to the carrier shell.

Of course, the foregoing description is that of certain features, aspects and advantages of the present invention, to which various changes and modifications can be made without departing from the spirit and scope of the present invention. Moreover, the glenoid prosthesis need not feature all of the objects, advantages, features, and aspects discussed above. Thus, for example, those skilled in the art will recognize that the invention can be embodied or carried out in a manner that achieves or optimizes one advantage or a group of advantages as taught herein without necessarily achieving other objects or advantages as may be taught or suggested herein. In addition, while a number variations of the invention have been shown and described in detail, other modifications and methods of use, which are within the scope of this invention, will be readily apparent to those of skill in the art based upon this disclosure. It is contemplated that various combinations or sub-combinations of these specific features and aspects of embodiments may be made and still fall within the scope of the invention. According, it should be understood that various features and aspects of the disclosed embodiments can be combined with or substituted for one another in order to form varying modes of the discussed glenoid prosthesis.

### LIST OF REFERENCE NUMERALS

10 Humerus nail
11 Bearing structure
12 Glenoid prosthesis
13 Bearing surface
14 Bearing surface
15 Scapula (shoulder blade)
16 Joint ball
17 Anchoring peg
18 Glenoid prosthesis
19 Carrier shell
20 Bearing structure
21 Bearing surface
22 Medial side
23 Anchoring ridge
24 Ridge expansion
25 Circumferential rim
26 Floor
27 Catch projection

What is claimed is:

1. A glenoid prosthesis, comprising:
a bearing, a lateral side of which defines a concave bearing surface; and
a carrier shell, a medial side of which comprises at least one anchoring element,
wherein the medial side is generally flat.

2. The prosthesis of claim 1, wherein the at least one anchoring element extends in an anterior/posterior (A-P) direction, so that in cement-free implantation the prosthesis can be inserted in the A-P direction.

3. The prosthesis of claim 1, wherein the at least one anchoring element comprises two ridges spaced apart from one another, each ridge having a free end extending in the anterior/posterior direction.

4. The prosthesis of claim 3, wherein the free ends of the anchoring ridges have ridge expansion portions.

5. The prosthesis of claim 4, wherein the ridge expansion portions have a cross-sectional shape selected from the group consisting of rectangular, trapezoidal, oval and cylindrical.

6. The prosthesis of claim 1, wherein the prosthesis is of modular construction and comprises a carrier shell and a bearing structure having said bearing surface, the carrier shell and bearing structure configured to fasten to each other.

7. The prosthesis of claim 6, wherein an intermediate layer of plastic is disposed between carrier shell and bearing structure.

8. The prosthesis of claim 7, wherein the intermediate layer is made of PE or PEEK.

9. The prosthesis of claim 6, wherein at least one of the bearing surface and the bearing structure comprises an oval or circular shape in plan view.

10. The prosthesis of claim 1, wherein the concave bearing surface comprises at least two spherical surfaces differing in radius.

11. The prosthesis of claim 1, wherein the concave bearing surface has a shape selected from the group consisting of an ellipsoid, a hyperboloid and a paraboloid surface.

12. The prosthesis of claim 6, wherein the bearing structure comprising the bearing surface is made of a material selected from the group consisting of plastic, polyethylene, ceramic, metal, and a composite thereof, the bearing structure fastened to the carrier shell either in situ or prior to implantation.

13. The prosthesis of claim 12, wherein the bearing structure is fastened to the carrier shell using a catch means in a manner selected from the group consisting of a force-fitting and a form-fitting manner.

14. The prosthesis of claim 1, wherein the carrier shell is made of a metal selected from the group consisting of titanium, titanium alloy, plastic, and a plastic-fiber composite.

15. The prosthesis of claim 1, wherein the medial side is roughened.

16. The prosthesis of claim 15, wherein the medial side is coated to facilitate bone growth onto the carrier shell.

17. The prosthesis of claim 1, wherein at least one of the medial side and the at least one anchoring element comprises recesses thereon to improve a connection between the prosthesis and the receiving bone via bone growth.

18. The prosthesis of claim 1, wherein the carrier shell comprises means for the fixation of spherical heads thereon.

19. The prosthesis of claim 1, wherein the medial side is completely planar.

20. A glenoid prosthesis, comprising:
a lateral bearing surface and at least one anchoring element on a medial side of the prosthesis;
wherein the at least one anchoring element comprises a ridge extending generally in an anterior-posterior direction and an enlarged portion at a free end thereof.

21. The prosthesis of claim 20, comprising a carrier shell comprising the at least one anchoring element and a bearing structure comprising the lateral bearing surface, wherein the carrier shell and the bearing structure are fixed to each other.

22. The prosthesis of claim 20, wherein the bearing surface is generally concave.

23. The prosthesis of claim 20, wherein the medial side comprises a pair of anchoring elements.

24. The prosthesis of claim 20, wherein the enlarged portion of the at least one anchoring element is cylindrical.

25. The prosthesis of claim 20, wherein the medial side of the prosthesis is generally flat.

26. The prosthesis of claim 20, wherein the lateral bearing surface is ball-shaped to engage a joint surface on a humerus side.

27. A method for implanting a glenoid prosthesis, comprising:
positioning a cutting guide on an anterior surface of the glenoid of a scapula;
removing bone from the scapula at the glenoid to a subchondral position to form a generally flat surface;
positioning a drill guide on the scapula;
drilling at least one bore in the scapula in generally an anterior-to-posterior direction;
forming at least one slot in communication with the at least one bore, said slot extending to the generally flat surface; and
attaching the glenoid prosthesis to the scapula, wherein the glenoid prosthesis includes a generally flat medial surface and at least one anchoring element projecting from the medial side and extending in generally an anterior-posterior direction, wherein the glenoid prosthesis is attached to the scapula by inserting the at least one anchoring element into the bore and the slot in an anterior-to-posterior direction.

28. The method of claim 27, wherein the at least one anchoring element comprises a ridge and an expanded portion at its free end, wherein the expanded portion is inserted into the bore and the ridge is inserted into the slot.

29. The method of claim 27, wherein drilling the at least one bore includes drilling two bores through apertures in the drill guide, and wherein the glenoid prosthesis comprises two anchoring elements.

30. The method of claim 29, wherein forming at least one slot includes forming two slots through elongated slots on the drill guide, said elongated slots communicating with the bores in the drill guide.

31. The method of claim 27, wherein positioning the cutting guide includes attaching the cutting guide to the scapula with a k-wire.

32. The method of claim 27, wherein positioning the drill guide comprises attaching the drill guide to the scapula with a k-wire.

33. A method for implanting a glenoid prosthesis, comprising:
forming a generally flat surface on a lateral side of a patient's scapula; and
attaching a glenoid prosthesis to the patient's scapula, the glenoid prosthesis having a generally flat surface on a medial side thereof to engage the generally flat surface on the lateral side of the patient's scapula, at least one anchoring element attaching the prosthesis to the scapula, and a bearing surface on a lateral side thereof.

34. The method of claim 33, wherein the glenoid prosthesis is attached to the scapula via at least one bore drilled into the scapula in generally an anterior-posterior direction.

35. A method for implanting a glenoid prosthesis, comprising:
forming at least one bore in generally an anterior-posterior direction in a patient's scapula, said bore communicating with a lateral side of the patient's scapula;
attaching a glenoid prosthesis to the patient's scapula, the prosthesis having at least one anchoring element on a medial side thereof, wherein the prosthesis is attached by inserting the anchoring element through the at least one bore such that the medial side of the glenoid prosthesis engages the lateral side of the patient's scapula.

36. The method of claim 35, wherein the at least one anchoring element comprises a ridge and an expanded portion at a free end thereof, the expanded portion fitting within the bore and the ridge fitting with a slot communicating between the bore and the lateral side of the patient's scapula.

37. The method of claim 35, further comprising resecting bone on the lateral side of the patient's scapula to form a substantially flat surface, and wherein the medial side of the glenoid prosthesis is substantially flat.
